(19) United States
(12) Patent Application Publication
(10) Pub. No.: US 2008/0249141 A1
(43) Pub. Date: Oct. 9, 2008

(54) Co-therapy with and combinations of statins and 1,4-dihydropyridine-3,5-dicarboxydiesters

(76) Inventor: Nageswara R. Palepu, South Hampton, PA (US)

Correspondence Address: Irving M. Fishman;c/o Cohen Tauber Spievack & Wagner, Suite 2400, 420 Lexington Avenue, New York, NY, 10170, US

(21) Appl. No.: 12/082,038

(22) Filed: Apr. 7, 2008

Related U.S. Application Data
(60) Provisional application No. 60/922,208, filed on Apr. 6, 2007.

(51) Int. Cl. A61K 31/4422

(52) U.S. Cl. 514/356

(57) ABSTRACT

Co-therapy of an 1,4-dihydropyridine-3,5-dicarboxyldiester and a statin is disclosed along with fixed combinations thereof. Amorphous atorvastatin hemicalcium salt and amlodipine besylate are preferred as the various components.

### CROSS-REFERENCE TO RELATED APPLICATIONS

This application claims the benefit of U.S. provisional Application Ser. No. 60/922,208, file Apr. 6, 2007

### STATEMENT REGARDING FEDERALLY SPONSORED RESEARCH OR DEVELOPMENT

Not Applicable

### FIELD OF THE INVENTION

The present invention relates to the field of statin therapeutic agents; to the field of 1,4-dihydropyridine-3,5-dicarboxydiesters; to combination therapy utilizing them together, either as separate administration of separate formulations or together as a single formulation. The invention further relates to improved methods of reducing cardiac risks due to cholesterol and triglycerides and high blood pressure by utilizing these agents in co-therapy as compared to use of one of these entities by themselves (inclusive of additive results which cannot be achieved by monotherapy at maximum tolerated dosages as well as synergistic results). The invention further relates to combination therapy which allows for reduction of the dosages of the individual agents below those levels at which they would be used in monotherapy to achieve the same or substantially the same results.

### BACKGROUND OF THE INVENTION

Various statins have been found to be effective HMG-CoA reductase inhibitors. Statins that are currently available for treating hyperlipidemia and/or hypercholesterolemia include atorvastatin (Lipitor® from Pfizer), simvastatin (Zocor® from Merck), pravastatin (Pravachol® from Bristol Myers Squibb), fluvastatin (Lescol® from Novartis), lovastatin (Mevacor® from Merck), and rosuvastatin (Crestor® from AstraZeneca). The second component of the present invention, is a 1,4-dihydropyridine-3,5-dicarboxydiester which are known to be antihypertensive agents.

### OBJECTS OF THE INVENTION

It is therefore an object of the invention to provide a method of enhancing the effectiveness of statins by administering to a patient in need thereof co-therapy which includes at least one statin in combination with at least one 1,4-dihydropyridine-3,5-dicarboxydiester.

It is another object of the invention to provide a composition comprising at least one statin and at least one 1,4-dihydropyridine-3,5-dicarboxydiester.

It is still another object of the invention to provide a synergistic composition comprising at least one statin and at least one 1,4-dihydropyridine-3,5-dicarboxydiester.

Yet another object of the invention is to provide a method of achieving a reduction in cholesterol and/or triglycerides and improve blood pressure control in a patient in need thereof that is in excess of such reductions achievable with monotherapy with either a statin or the 1,4-dihydropyridine-3,5-dicarboxydiester.

Still another object of the invention is to provide a statin co-therapy with at least one 1,4-dihydropyridine-3,5-dicarboxydiester where the statin is atorvastatin or a pharmaceutically acceptable salt thereof and/or the 1,4-dihydropyridine-3,5-dicarboxydiester is amlodipine.

Yet another object of the invention is to provide a statin/1,4-dihydropyridine-3,5-dicarboxydiester cotherapy further having at least one additional antihypertensive agent.

Still another object of the invention is to provide a statin/1,4-dihydropyridine-3,5-dicarboxydiester cotherapy further having at least one anti-obesity agent.

Even further object of the invention will be apparent to those of ordinary skill in the art.

### BRIEF SUMMARY OF THE INVENTION

These and other objects of the invention can be achieved by treating patients in need of cholesterol and/or serum triglyceride reduction or control and/or blood pressure reduction or control with co-therapy comprising at least one statin and at least one 1,4-dihydropyridine-3,5-dicarboxydiester. Preferably, the co-therapy is via a single dosage form having both the statin and at least the 1,4-dihydropyridine-3,5-dicarboxydiester component in the same dosage form.

### BRIEF DESCRIPTION OF THE SEVERAL VIEWS OF THE DRAWING

Not Applicable

### DETAILED DESCRIPTION OF THE INVENTION

The present invention is a combination of a statin and at least one 1,4-dihydropyridine-3,5-dicarboxyldiester, whether in a single dosage form or administered in separate dosage forms each having one of the two active agents (statin and 1,4-dihydropyridine-3,5,-dicarboxydiester), either simultaneously, sequentially, or at different times of the day. In addition to these agents mentioned above, additional active agents can be optionally added to the co-therapy regimen, whether as additional standalone products or as fixed combination products with any or all of the other active agents. Whenever an active agent is referred to herein, it includes the free compound named and its various pharmaceutically acceptable salts. Mention of the compound name, without reference to polymorphic form or crystallinity or lack of crystallinity includes amorphous and crystalline farms of any kind. Mention of the compound name without reference to solvate or non-solvate includes hydrates, anhydrous forms, other solvates, unsolvated forms, and mixed solvates (a hydrate being a solvate where the solvent molecule is water).

In the simplest form of the invention the at least one 1,4-dihydropyridine-3,5-dicarboxyldiester component and the statin component are in different dosage forms. In this aspect, the currently marketed forms of at least one 1,4-dihydropyridine-3,5-dicarboxyldiester and any of the statins is suitable and they may be used in amounts that range from the below the minimally effective amounts as set forth in their respective labeling as of the filing date of this application (i.e., taking benefit of the synergistic results of the invention) to a maximum of their respective maximum tolerated dosages as indicated in their respective labeling (generally not greater than twice the maximum recommended amounts in their respective current labeling) as of the filing date of the present application (obtaining results that would not be achievable with either entity as monotherapy even beyond the maximum tolerated dose). Preferably, the maximum tolerated dosage of the individual agents is not used and the preferred maximum amount of each agent is within the maximum recommended dosages in their respective labeling as of the filing date of the present application. There is no set ratio of one component to the other within the above amounts that is not or should not be considered for use, all of them being within the current invention. For the respective compounds that are not currently marketed, the range of dosages for consideration in the present invention should be that amount which gives approximately equal therapeutic responses on average to its closest marketed related compound in at least one indication for its closest marketed related compound as of the filing date of his application. Thus, if an unmarketed “atorvastatin-like drug” used as the statin, its range of dosages for the present invention should be based on either atorvastatin (currently marketed in the US) or to a more closely related statin that is currently marketed elsewhere in the world. Of course, if the more closely related statin is marketed elsewhere in the world (i.e., other than the US) as of the filing date of the present application but not in the US, then the dosage should be calculated based on that marketed labeling. Where the US dosage range and the dosage range from other countries differ, the lowest minimum and highest maxium (not necessarily in the same label) should be considered as the “currently marketed range”. Similar guidelines should be used for the dose calculation of the 1,4-dihydropyridine-3,5-dicarboxydiesters. Additional active agents that are desirable to coadminister and are included in the co-therapy or co-formulation should generally be used in the dosage ranges recommended in their respective labeling when those additional actives are otherwise used as standalone therapy.

The statins belong to a group of compounds that have the following formula I

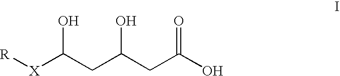

where X is straight or branched —(CH₂)ₘ— or —CH═CH—, preferably —(CH₂)ₘ—; with m being 0-4 (preferably 1) or its corresponding lactone of formula II

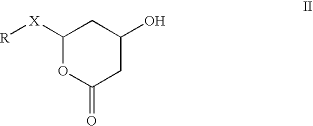

where R in each case is a 5-6 membered monocyclic or 9-10 membered bicyclic group which may be substituted with a variety of substituents. For purposes of the present invention, the term “statin also includes (unless specifically restricted otherwise or the context requires restriction) the pharmaceutically acceptable salts and esters of the acid group shown in Formula I above. Typical statins that are commercially available include: atorvastatin, fluvastatin, lovastatin, pravastatin, rosuvastatin, and simvastatin.

R in formula I may be selected from the group of formulas III, IV, V, and VI; where formula III is

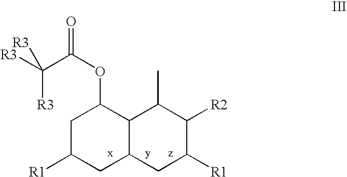

where R1-bond x-bond y-bond z-R1 represents R1—C*H—CH═C*—CH═C*—R1,

### R1—C*H—CH═C*—CH₂C*H—R1, R1—C*H—CH₂—C*═CH—C*H—R1, or

R1—C*H—CH₂—C*H—CH═C*—R1; where * indicates a bond to the rest of the structure (in other words either (1) one of the bonds x, y, and z is a double bond or (2) y is a single bond and both x and z are double bonds);
each R1 being independently selected form H, OH, or alkyl of 1-4 carbon atoms (preferably of 1 carbon);
R2 being selected form H or alkyl of 1-4, preferably 1, carbon atom;
each R3 being independently selected from H and alkyl of 1-4 carbons, preferably of 1 carbon;

where formula IV is

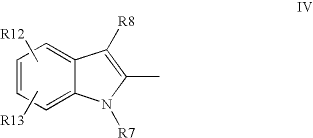

in which one of R7 and R8 is a phenyl ring optionally having from 1-3 substituents independently selected from selected from alkyl of 1-4 carbonas, alkoxy of 1-4 carbons, halogen (preferably fluoro or chloro), phenoxy, and benzyloxy; the other of R7 and R8 is a primary or secondary alkyl of 1-5 carbons; and each of R12 and R13 is independently selected from H, straight or branched chain alkyl of 1-4 carbons, straight or branched alkoxy of 1-4 carbons, cycloalkyl of 3-6 carbons, trifluoromethyl, fluoro, chloro, phenoxy and benzyloxy;

where formula V is

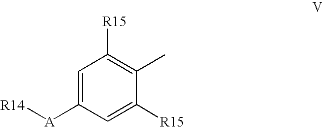

where A is S, —SO₂—, or N, the N being optionally substituted by straight or branched alkyl of 1-5 carbon atoms (preferably methyl);
R14 is selected from (1) alkyl of 1-3 carbons (preferably methyl), optionally substituted by 1-3 substituents selected from halogen, amino, and/or cyano, (2) an aromatic group of 6-12 carbons optionally substituted by 1-3 substituents selected form alkyl of 1-3 carbons, halogen, amino, or cyano, or (3) alkyl of 1-3 carbons (preferably methyl), optionally substituted by 1-3 substituents independently selected from an aromatic group of 6-12 carbons which is further optionally substituted by 1-3 substituents selected form alkyl of 1-3 carbons, halogen, amino, or cyano;
each of R15 is independently selected from (1) H, (2) alkyl of 1-3 carbons optionally substituted by halogen, amino, and/or cyano, and (3) an aromatic of 6-12 carbons (preferably phenyl) optionally substituted by alkyl, halogen (preferably fluoro), and/or amino;

where formula VI is

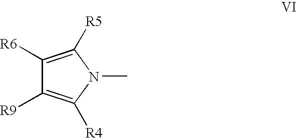

where R4 is selected from straight or branched alkyl of 1-6 carbons, cycloalkyl of 3-6 carbons, and trifluoromethyl;
R5 is selected from 1-naphthyl, 2-naphthyl, cyclohexyl, norbornyl, or phenyl (optionally substituted with fluorine, chlorine, bromine, hydroxyl, trifluoromethyl, alkyl of 1-4 carbons, alkoxy of 1-4 carbons, or alkanoyloxy of from 2-8 carbons); either of R6 or R9 is —CON(R10)(R11) in which R10 and R11 are each independently selected from hydrogen, alkyl of 1-6 carbons, or phenyl optionally substituted with fluorine, chlorine, bromines, cyano, trifluoromethyl and/or carboalkoxy of 3-8 carbon atoms;
and the other of R6 and R9 is selected from hydrogen, alkyl of 1-6 carbon atoms, cyclopropyl, cyclobutyl, cyclopentyl, cyclohexyl, or phenyl, which phenyl is optionally substituted with fluorine, chlorine, broine, hydroxyl, trifluoromethyl, alkyl of 1-4 carbons, alkoxy of 1-4 carbons, and/or alkanoyloxy of 2-8 carbons;

Atorvastatin and atorvastain-like drugs are of formula VI above and are described more specifically, including the manner of making and using them, in one or more of U.S. Pat. Nos. 4,681,893; 5,273,995; 5,686,104; 5,969,156; and 6,126,971, all of which are incorporated herein by reference in their entireties. In some embodiments, the atorvastatin or atorvastatin-like drug is in the form of its calcium salt. “Atorvastatin” as the free compound is specifically the compound (βR, δR)-2-(4-fluorophenyl)-beta,delta-dihydroxy-5-(1-methylethyl)-3-phenyl-4-[(phenylamino)carbonyl]-1H-pyrrole-1-heptanoic acid.

Simvastatin and simvastatin-like drugs belong to formula III above and are described more specifically, including the manner of making and using them, in one or more of U.S. Pat. Nos. 4,444,784; RE36481; and RE36520, all of which are incorporated herein by reference in their entireties.

Pravastatin and pravastatin-like drugs belong to formula III above and are described more specifically, including the manner of making it and using it, in one or more of U.S. Pat. Nos. 4,346,227; 5,030,447; 5,180,589; and 5,622,985; all of which are incorporated herein by reference in their entireties.

Fluvastatin and fluvastatin-like drugs belong to formula IV above and are described more specifically, including the manner of making and using them, in one or more of U.S. Pat. Nos. 5,354,772; and 5,356,896, each of which is incorporated herein by reference in their entireties.

Lovastatin and lovastatin-like drugs belong to formula III above and are described more specifically, including the manner of making and using them, in U.S. Pat. No. 4,231,938, which is incorporated herein by reference in its entirety.

Rosuvastatin and rosuvastatin-like drugs belong to formula V above and are described more specifically, including the manner of making and using them, in one or more of U.S. Pat. Nos. 6,316,460; 6,589,959; and RE 37,314, all of which are incorporated herein by reference in their entireties.

The 1,4-dihydropyridine-3,5-dicarboxyldiester for use in the present invention is selected from those having the following formula IX

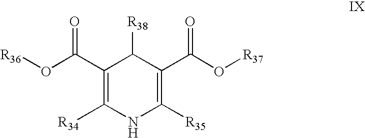

where R34 and R35 are each independently selected from alkyl of 1 to 4 carbon atoms (preferably methyl), alkyl of 1 to 4 carbon atoms having a oxygen substituent on any one of the carbon atoms thereof (preferably on the carbon adjacent to the dihydropyridine ring) (and also preferably a methyl group having an oxygen thereon) wherein the oxygen is further substituted by an alkylene bridge of 1 to four carbons which may be straight chain or branched, and which bound to an unsubstituted amino or a mono or di substituted amino, each of the amino substituents being independently selected from alkyl of 1 to 4 carbons, 2-(alkoxy of 1 to 4 carbons)ethyl, cyclopropyl, methyl, benzyl, of —(CH₂)ₘCOR³⁹ where m is 1, 2, or 3, and R³⁹ is hydroxyl, alkoxy of 1 to 4 carbons, of —NR⁴⁰R⁴¹ with R⁴⁰ and R⁴¹ each being independently hydrogen or alkyl of 1 to carbons;
R³⁶ and R³⁷ each being independently alkyl of 1 to 4 carbons, 2-methoxyethyl, 2-(N-methyl-benzylamino)ethyl;
R³⁸ is an aryl groups of 6 to 14 carbons (preferably phenyl) or a heteroaryl group having up to 14 ring members in up to 3 fused rings and up to 4 nitrogen heteroatoms and/or up to 2 oxygen heteroatoms and/or up to 2 sulfur heteroatoms, which aryl and heteroaryl groups are optionally substituted with up to three substituents selected from nitro, halo, alkyl of 1 to 4 carbons, alkoxy of 1 to 4 carbons, trifluoromethyl, or hydroxyl; and pharmaceutically acceptable salts thereof. Preferably, R³⁴ is methyl, R³⁵ is methyl or 2-aminoethoxymethyl, R³⁶ ismethyl, ethyl, or isopropyl, R³⁷ is methyl, ethyl, isopropyl, 2-(N-methylbenzylamino)ethyl, or 2-methoxyethyl, and R³⁸ is (a) phenyl optionally substituted by 1 or 2 halogen (preferably chloro) or nitro, or (b) benzofuraazanyl. Preferred 1,4-dihydropyridine-3,5-dicarboxyldiesters for use in the present invention are the currently marketed (in the US 1,4-dihydropyridine-3,5-dicarboxyldiesters, namely, amlodipine (Norvasc® from Pfizer), felodipine (Plendil® from AstraZeneca), isradipine (Dynacirc® from Reliant), nicardipine (Cardene® from PDL Pharma), nifedipine (Procardia® from Pfizer) nimodipine (Nimotop® from Bayer), and nisoldipine (Sular® from Sciele Pharma). More highly preferred is amlodipine or a pharmaceutically acceptable salt thereof. The besylate salt and tartrate salt of amlodipine are particularly preferred, with the besylate salt of amlodipine being the most highly preferred. These are discussed in further detail, including methods of making and using the compounds, in U.S. Pat. No. 4,572,909, U.S. Pat. No. 4,879,303, U.S. Pat. No. 4,803,081, U.S. Pat. No. 4,816,263, U.S. Pat. No. 4,946,687, U.S. Pat. No. 4,950,486, U.S. Pat. No. 5,030,456, U.S. Pat. No. 5,198,226, U.S. Pat. No. 4,892,741, U.S. Pat. No. 5,264,446, and U.S. Pat. No. 4,892,741, each of which is incorporated herein in its entirety by reference.

Optional additional active agents that may be combined with the binary co-therapy of the present invention include, without limitation, other antihypertensive (such as non-1,4-dihydropyridine-3,5-dicarboxydiester antihypertensives and diuretics) and anti-obesity agents (such as without limitation or list at and/or sibutramine), among others.

Of the above statins, atorvastatin, fluvastatin, lovastatin, pravastatin, simvastatin, and rosuvastatin, or pharmaceutically acceptable salts thereof (or the lactone or the non-lactone variants thereof as applicable) are preferred, in part because they are in commercial medical use. Of these, atorvastatin, its pharmaceutically acceptable salts, and the lactone version thereof is more highly preferred. Of the atorvastatin salts, any of the pharmaceutically acceptable salts may be used, but the amino acid, sodium and calcium salts are preferred with the calcium salt being more highly preferred.

Atorvastatin is currently recommended in its current US labeling for cholesterol reduction at doses of 10 mg to 80 mg once daily. For purposes of the present invention, it can be used at dosages as low as 2.5 mg up to 160 mg once daily or in divided doses, generally up to 80 mg once daily or in divided doses.

Lovastatin is currently recommended to be administered in its current US labeling in amounts of 10 mg to 80 mg once daily or in 2 divided doses. For purposes of the present invention lovastatin can be used at doses as low as 2.5 mg up to 160 mg once daily or in divided doses, generally up to 80 mg once daily or in divided doses.

Fluvastatin is recommended to be administered in its current US labeling in doses of from 20 mg to 80 mg once daily or in divided doses. The present invention allows for fluvastain to be doses at 5 mg daily up to 160 mg once daily or in divided doses, generally up to 80 mg daily in single or divided doses.

Pravastatin is recommended for administration in its current US marketed label in amounts of 10 mg to 80 mg once daily. The present invention allows for the use of pravastatin at a dose as low as 2.5 mg daily up to 160 mg once daily or in divided doses, generally up to 80 mg daily.

Simvastatin is recommended in its current US label at doses of 5-80 mg once daily. The present invention permits dosing of simvastatin at 1.25 mg daily up to 160 mg once daily or in divided doses, generally up to 80 mg daily.

Rosuvastatin, when used for hypercholesterolemia control is dosed at 5 mg to 40 mg, once daily. The present invention permits dosing of rosuvastatin at about 1 mg daily to about 80 mg once daily or in divided doses, generally up to 40 once daily or in divided doses.

The 1,4-dihydropyridine-3,5-dicarboxyldiester can be used in the present invention in amounts which are from about ¼, preferably ½, the minimum effective dose in the current label of the currently marketed 1,4-dihydropyridine-3,5-dicarboxyldiesters to the maximum tolerated dose in their respective current labels. For those compounds that are not marketed in the US, but marketed outside the US as of the filing date of this application, similar dosage guidance can be found in that labeling. If a product is marketed in multiple countries and the minimum and maximum in the various labels differ, the invention is intended to include a dosage range from ¼, preferably ½, the lowest minimum to the maximum tolerated dose, preferably to highest maximum in such labels, whether the minimum and maximum chosen is in the same or different labels. Where a 1,4-dihydropyridine-3,5-dicarboxyldiester is within the above formula IX, but not currently marketed, the minimum amount for the present invention is ¼, preferable ½, of an amount which is approximately equipotent with the minimum label recommend amount of the closest marketed compound thereto and the maximum is to be taken as the maximum tolerated dose thereof, preferably that quantity of the unmarketed drug which gives an approximate equipotent response to the maximum label recommended dose (if lower) of the closest marketed compound thereto.

The ratio of the 1,4-dihydropyridine-3,5-dicarboxyldiester to the statin can be any ratio that permits the 1,4-dihydropyridine-3,5-dicarboxyldiester and the statin to be administered within the ranges set forth above; however, most preferable for ease of use of the currently marketed standalone products are ratios which use a currently marketed dosage form of each active. In a most highly preferred combination, the statin is atorvastatin or a pharmaceutically acceptable salt thereof and the 1,4-dihydropyridine-3,5-dicarboxyldiester is amlodipine or a pharmaceutically acceptable salt thereof. For purposes of exemplification only, a 10 mg, 20 mg, 40 mg, or 80 mg dosage form of atorvastatin as currently marketed may be paired with any of the 2.5 mg, 5 mg, and 10 mg dosage forms of amlodipine. These atorvastatin and amlodipine binary combinations (except for 2.5 mg amlodipine with 80 mg atorvastatin) are currently marketed by Pfizer as combination products of amlodipine besylate and atorvastatin calcium. With respect to this particular binary pair, the present invention differs therefrom in that the atorvastain therein is crystalline atorvastatin calcium, while that in the present invention is a physically and chemically stable non-crystalline atorvastatin, which is prepared in accordance with the methods set forth in Pending U.S. application Ser. No. 11/282,507, filed Nov. 18, 2005 or in accordance with procedures set forth in PCT/U.S.07/04629, filed Feb. 20, 2007, each of which is incorporated herein in its entirety by reference. Furthermore, the present invention formulation of atorvastatin (whether as a standalone product or as a fixed combination with amlodipine differs significantly from the atorvastatin formulation and fixed combination marketed by Pfizer. A significant advantage of the present invention is that it allows for the preparation of non-infringing versions the atorvastatin component of the Pfizer combination product while patents claiming the crystalline atorvastatin calcium are still in force. It also allows for the avoidance of such surfactants as Tweens and inorganic salt stabilizers such as calcium carbonate in the atorvastatin containing component.

Sample fixed combination dosages are set forth below for atorvastatin and currently US marketed 1,4-dihydropyridine-3,5-dicarboxyldiesters. Similar ratios for the other marketed statins when used in co-therapy with 1,4-dihydropyridine-3,5-dicarboxyldiesters will be apparent to those of ordinary skill. Dosages for other statins and 1,4-dihydropyridine-3,5- dicarboxyldiesters that are not the specific ones set forth above but are within the formulas above can be calculated as:
unmarketed statin compound minimum for the invention=¼×amount which is of approximately equal therapeutic effect as that of the minimum recommended amount of the closest marketed statin
unmarketed statin compound maximum for the invention=maximum tolerated dose of the unmarketed statin compound
unmarketed 1,4-dihydropyridine-3,5-dicarboxyldiester compound minimum for the invention=¼×amount which is of approximately equal therapeutic effect as that of the minimum recommended amount of the closest marketed 1,4-dihydropyridine-3,5-dicarboxyldiester
unmarketed 1,4-dihydropyridine-3,5-dicarboxyldiester compound maximum for the invention=maximum tolerated dose of the unmarketed 1,4-dihydropyridine-3,5-dicarboxyldiester compound.

Sample (non-limiting) combination dosages (free combination of marketed dosage forms)*:

| | 1,4-dihydro- | | | |
|---|---|---|---|---|
| | pyridine- | | | |
| | 3,5-dicarboxyl- | | Atorvastatin | |
| Particular dihydropyridine | diester | | Dose | |
| Amlodipine or felodipine | 2.5 | mg | 10 | mg |
| Amlodipine or felodipine | 2.5 | mg | 20 | mg |
| Amlodipine or felodipine | 2.5 | mg | 40 | mg |
| Amlodipine or felodipine | 2.5 | mg | 80 | mg |
| Amolodipine, felodipine, or isradipine | 5 | mg | 10 | mg |
| Amolodipine, felodipine, or isradipine | 5 | mg | 20 | mg |
| Amolodipine, felodipine, or isradipine | 5 | mg | 40 | mg |
| Amolodipine, felodipine, or isradipine | 5 | mg | 80 | mg |
| Amolodipine, felodipine, or isradipine, | 10 | mg | 10 | mg |
| nifedipine, or nisoldipine | | | | |
| Amolodipine, felodipine, or isradipine, | 10 | mg | 20 | mg |
| nifedipine, or nisoldipine | | | | |
| Amolodipine, felodipine, or isradipine, | 10 | mg | 40 | mg |
| nifedipine, or noisoldipine | | | | |
| Amolodipine, felodipine, or isradipine, | 10 | mg | 80 | mg |
| nifedipine, or nisoldipine | | | | |
| Nicardipine, or nifedipine, or nisoldipine | 20 | mg OD | 10 | mg OD |
| Nicardipine, or nifedipine, or nisoldipine | 20 | mg | 20 | mg BID |
| Nicardipine, or nifedipine, or nisoldipine | 20 | mg | 40 | mg TID |
| Nicardipine, or nifedipine, or nisoldipine | 20 | mg | 80 | mg OD |
| Nicardipine, nimodipine, or nisoldipine | 30 | mg | 10 | mg BID |
| Nicardipine, nimodipine, or nisoldipine | 30 | mg | 20 | mg OD |
| Nicardipine, nimodipine, or nisoldipine | 30 | mg | 40 | mg OD |
| Nicardipine, nimodipine, or nisoldipine | 30 | mg | 80 | mg BID |
| Nisoldipine | 40 | mg | 10 | mg |
| Nisoldipine | 40 | mg | 20 | mg |
| Nisoldipine | 40 | mg | 40 | mg |
| Nisoldipine | 40 | mg | 80 | mg |

*OD = once daily; BID = twice daily; TID = three times daily

Fixed combination dosage forms can be prepared in any manner known in the art and are especially prepared from the materials that are utilized in the formulation of the standalone single active agent corresponding products. They may be made by blending the active agents together in a single blend, or preparing pre-blends of less than all of the active agents and forming each into separate granulations for blending together, or the actives can be individually prepared into beads for blending and filling into capsules or compression into tablets. In other formats, one or more of the active agents can be formulated as a separate portion of the dosage form as in the case of bi-layered, tri-layered, or multilayered tablets. Those of ordinary skill in the art will be aware of further variations on the theme. Particularly advantageous formulations for atorvastatin or for atorvastatin containing fixed combinations are set forth more fully below.

In addition to the above, it should be noted that one or more of the active agents can be administered by alternative routes of administration, i.e., non-oral routes for any of the actives. Thus, an oral 1,4-dihydropyridine-3,5-dicarboxyldiester combined with a transdermal administration of the statin, for example, is also within the present invention. Those of ordinary skill will be aware of further alternate routes by which the statin and other 1,4-dihydropyridine-3,5-dicarboxyldiesters can be administered. An exception to this is with certain anti-obesity drugs that may be optionally present, because certain agents of this type act in the intestinal tract and must be administered in some fashion to the intestinal tract. Those of ordinary skill in the art would know when this limitation on the administration route is involved and with which agents.

In each of the above embodiments, whether separate agents in separate dosage forms, or fixed combinations, one or more further active agents can also be added to the co-therapy regimen. These further agents can be added in free combination with the above or may also be in fixed combination with one or more of the other agents. For example, in a three active agent scenario, (a) each of the active agents 1, 2, and 3 may be used in free combination, or (b) agents 1 and 2 may be in fixed combination with each other and used in free combination with agent 3, or agents 1 and 3 may be used in fixed combination with each other and used in free combination with agent 2 or agents 2 and 3 may be in fixed combination with each other and used in free combination with agent 1 or (c) all of agents 1, 2, and 3 are in fixed combination with each other. Those of ordinary skill in the art will appreciate the various alternatives when still further active agents are added to the co-therapy.

Inactive agents which can be used are any of those that are compatible with the active agents that are in contact therewith and are pharmaceutically acceptable. These are generally known in the art (both components and relative amounts and specifically indicated in the various patents set forth herein, all of which are incorporated herein in their entirety by reference). These typically include, without limitation, active agent stabilizers (inclusive of chemical stabilizers and physical stabilizers, etc.), diluents, binders, disintegrants, surfactants, lubricants, glidants, and coating materials. Any of the inactive agents present in the currently marketed products containing the respective active agent may be used for that component of the fixed combination products of the present invention and unless there is an incompatibility that results with the other active agents in the invention fixed combinations, may be used in intimate contact with the other active agent as well. Notwithstanding the above, certain preferred formulations utilize non-crystalline active agents prepared in accordance with methods set forth in Pending U.S. application Ser. No. 11/282,507, filed Nov. 18, 2005 and procedures set forth in PCT/U.S.07/04625, filed Feb. 20, 2007.

Where single granulations contain both active agents, then the inactives need to be compatible with both active agents, Since coating materials are not in intimate contact with the active agents, they may, in some instances have some incompatibilities with the active agents, and if so, then it is preferably to have an intermediary barrier coating that separates the incompatible coating components form the remainder, but if acceptable formulation stability in the absence of such intermediary barrier is obtained, the barrier layer need not be used. Those of ordinary skill will be able to select the appropriate coating materials based on simple testing or knowledge already available in the art.

Typical preferred inactive agents include, without limitation, bulking agents (for example without limitation, mono and disaccharides (such as dextrose, lactose, sucrose, etc.), sugar alcohols (such as mannitol, xylitol, sorbitol. etc.) and other bulking agents (such as microcrystalline cellulose, dicalcium phosphate, tricalcium phosphate, etc.)), surfactants (such as polyethyleneglycols, polyethylene glycol/polypropylene glycol block or random compolymers, Tweens, Vitamin E TPGS, Tween surfactants, Brij surfactants, fatty alkyl sulfates, fatty alkyl sulfonates, polyethoxylated fatty alkyl sulfates, polyethoxylated fatty alkyl sulfonates, etc.), binders (such as hydroxypropylcellulose, hydroxypropylmethylcellulose, hydroxyethylcellulose, povidone, carboxymethylcellulose, sodium carboxymethylcellyulose, etc.), disintegrants and superdisintegrants (such as povidone, crospovidone, croscarmellose sodium, sodium starch glycollate, etc), alkalinizing salts such as (alkali metal or alkaline earth metal salts of carbonate or bicarbonate or silicate, alkaline earth metal hydroxide, magnesiumaluminum silicate, magnesium aluminum hydroxide, etc.), lubricants and glidants (such as alkali metal or alkaline earth metal salts of fatty acids, silicon dioxide, talc, etc.), and typical coating agents known in the art. The typical coating agents can be mere film coatings that do not alter dissolution profiles (for example, without limitation, those available under the OPADRY name), those that delay release that are either pH dependent or pH independent, and those that impart controlled or sustained release. Each of the inactive agents can vary over wide ranges in terms of the percent of the formulation that they make up and is in part dependent upon the amount of active agent being administered and the particular dissolution profile being sought. A highly preferred formulation is set forth in the Examples, but a wide range of other compositions are suitable as well.

Dosage form construction can be along the lines of single granulation, with one or both agents in the granule or one active agent intragranularly and one extragranularly, or one or both active agents can be coated or adsorbed onto or into a carrier particle. Alternatively, one or both active agents may be included into an oral-osmotic dosage form of the type that has become known as OROS formulations, many of which have been patented by ALZA Corporation in the 1970s and 1980s. Alternatively, bilayer or multilayer formulations may be prepared where the active agents may be in the same or different layers and the different layers may have similar or different physical functions with respect to release rates such as rapid swelling to allow for gastric retention of all or part of the dosage form in the stomach for release of one or more of the active agents in the stomach (such as for example, without limitation, those patented by Jagotech or by Depomed). A further alternative is to have a capsule dosage form (whether hard or soft) containing the various active agents either as granulates or in the form of minitablets, with or without extragrnaular inactive agents or extragranular active agent as well. Still other dosage form constructions for fixed combinations will be apparent to those of ordinary skill in the art.

In a particularly preferred embodiment, a statin active agent and a 1,4-dihydropyridine-3,5-dicarboxydiester active agent are blended with a superdisintegrant such as croscarmellose sodium or sodium starch glycollate and optionally microcrystalline cellulose. This blend is granulated with an aqueous solution or dispersion of a surfactant like material such as Vitamin E TPGS, which granules are then sieved and dried. The dried granules are then blended with a carrier such as lactose, microcrystalline cellulose, a disintegrant such as croscarmellose sodium or sodium starch glycollate, and either or both of a lubricant and glidant. The blend is then compressed into a single tablet. Alternatively, the statin and the 1,4-dihydropyridine-3,5-dicarboxdiester can be separately granulated using the same or different excipients and the two granulates are blended with the extragranular materials. Similarly, a portion or all of either of the active agents can be in the extragranular portion. Surprisingly, advantageously the above formulation (set forth in greater detail below) allows for the stain (a generally negatively charged moiety) and the 1,4-dihydropyridine-3,5-dicarboxydiester (generally a positive charged moiety) to be in intimate admixture. Note that the art is generally aware that there are incompatibilities between such charged active moieties.

Additional active agents can be added as and intragranulate component of either of the statin granulate or 1,4-dihydropyridine-3,5-dicarboxydiester granulate or the combined granulate, or if desired can be added extragranularly. Design choices such as the individual active agent pharmacokinetics will help guide the choice, but any arrangement is within the scope of the present invention. Generally, most active agents will be at least partially within one or morte of the granulates. Alternatively, the additional active agents may be formulated in their own granulates which are blended with the granulate or granulates containing one or both of the statin and the 1,4-dihydropyridinedicarboxydiester.

Additional alternate processes may include colyophilization of both of the two co-therapy active agents (with or without colyophilization with other optional active agents) with or without surfactant or solubilizer and with or without an internal disintegrant. The lyophilized blend is then mixed with bulking excipients and disintegrant, lubricated and compressed into tablets or filled into capsules. A binder can also be used in the colyophilization of the active agents.

Exemplary formulations are set forth in the examples appended hereto. Using the formulations in Example 1 there and the statin as the active agent alone as a base formulation (i.e. an 80 mg atorvastatin stand alone formulation, that is without the amlodipine or list at of the examples), the formulation can have the other required and/or optional active agents active agents added intragranularly by replacing a portion of the intragranular and/or extragranular microcrystalline cellulose and/or extragranular lactose or simply be added to the base composition intragranularly or extragranularly. The additional required active agents and/or optional active agents can be added alternatively as their own granulate or extragranularly as desired, generally by replacing a portion of the extragranular microcrystalline cellulose and/or lactose. When used extragranularly, they can be added in partial replacement of the extragranular microcrystalline cellulose and/or lactose, or simply added without replacement of any of the microcrystalline cellulose or lactose. In this manner, each of the 80 mg atorvastatin containing compositions can be obtained with the additional required and/or optional active agents of the co-therapy in fixed combinations thereof. For lower dose atorvastatin, one can either start with a proportional amount of the 80 mg atorvastatin base formulation mentioned above (i.e., ⅛th for a 10 mg formulation) or start with the base formulation set forth above except using a lesser amount of the atorvastatin (i.e., simply replace the 80 mg atorvastatin with 10 mg atorvastatin in the otherwise base formulation referred to above) and include the other active agents as indicated above concerning the 80 mg containing combinations. In each of these, the atorvastatin may be replaced by appropriate amounts of the other statins to arrive at formulations containing those statins. Furthermore, in each case, the microcrystalline cellulose and lactose can be replaced in whole or in part by other pharmaceutically acceptable bulking agents such as, without limitation, those as set forth previously, and the croscarmellose sodium and sodium starch glycolate can be in whole or part replaced by other pharmaceutically acceptable disintegrants, such as, without limitation, those as set forth above, and the magnesium stearate can be replaced in whole or part by other pharmaceutically acceptable lubricants and/or glidants, such as, without limitation, those as set forth above. In each of the formulations thus arrived at (which are the most preferred amounts), the ranges of the inactive components can vary from those derived from the above (to arrive at still preferred, but not most preferred amounts) as follows: the bulking agents can be +/−about 15% of the amounts otherwise arrived at; the disintegrants can be +/−about 15% of the amounts otherwise arrived at; the lubricants/glidants can be +/−about 2% of the amounts otherwise arrived at, and the TPGS component should be at a minimum of about 5 mg in any formulation and can vary up to about 40 mg in any formulation otherwise arrived at. Notwithstanding the above, even broader variations will be apparent to those of ordinary skill in the art once aware of the present invention.

The following examples, exemplify, but do not limit, the invention, which is limited only by the claims appended hereto.

### EXAMPLE 1

Exemplary tablet compositions of the invention using atorvastatin calcium as the statin active agent and amlodipine besylate as the 1,4-dihydropyridine-3,5-dicarbvoxydiester active agent, with and without or list at as a third active agent are presented below:

| | Composition | Composition | Composition |
|---|---|---|---|
| Ingredients | 1 | 2 | 3 |
| Intra-granular | | | |
| Atorvastatin Ca * | * | * | * |
| Amlodipine besylate ** | ** | ** | ** |
| Croscarmellose Sodium | 48 | 48 | 48 |
| Vitamin E TPGS | 20 | 20 | 20 |
| MCC PH 102 | — | — | 162.6 |
| Extra-granular | | | |
| Orlistat | — | 80 | 120 |
| Lactose Monohydrate | 292 | 292 | 292 |
| (Pharmatose DCL 11) | | | |
| MCC Avicel pH 102 | 269.67 | 269.67 | 87.07 |
| Sodium starch glycollate | 48 | 48 | 48 |
| Magnesium stearate | 7.2 | 7.2 | 7.2 |
| Coating | | | |
| Opadry | 23 | 23 | — |
| white | | | |
| Opadry | — | — | 23 |
| pink | | | |

* EQUIVALENT TO 80 MG ATORVASTATIN
** EQUIVALENT TO 5 MG AMLODIPINE

Method of Manufacture:

The atorvastatin calcium and croscarmellose sodium, amlodipine and MCC were sifted through a sieve and dry mixed for in a high shear mixer. Vitamin E TPGS was dissolved in warm water (at 70° C.-75° C. temperature) to get a clear solution and used to granulate the dry mixture above. The wet mass was passed through a sieve and dried at a product bed temperature of 45-50° C., and the dried granules were sized through #20 sieve, and then mixed with the extra-granular materials (other than the lubricant). Then the lubricant was added and the ingredients blended in a drum blender, and the blended material was tableted. The resulting tablets were then coated with the appropriate Opadry composition as indicated in the above table.

The composition 1 tablets above (80 mg atorvastatin/5 mg amlodipine) were tested for dissolution parameters. Dissolution testing is carried out in USP Type II (paddle) equipment at 50 rpm using either 900 ml of water or 0.1 N HCl containing 0.2% NaCl with quantification by HPLC. Comparison to Amlogard (a 5 mg amlodipine besylate tablet by Pfizer was also prepared using 900 ml of water as the dissolution media or 900 ml of 0.1N HCl containing 0.2% sodium chloride, with quantification by UV methods. The results are shown below:

Summary of Results (Atorvastatin) from the Invention (Water Media)

| | SAMPLE No. | | | | | | | |
|---|---|---|---|---|---|---|---|---|
| TIME | 1 | 2 | 3 | 4 | 5 | 6 | MEAN | RSD |
| 5 Minutes | 42.7 | 30.3 | 45.9 | 32.7 | 35.2 | 28.7 | 35.9 | 19.3% |
| 10 | 67.3 | 52.8 | 67.5 | 56.5 | 65.2 | 57.0 | 61.1 | 10.4% |
| Minutes | | | | | | | | |
| 15 | 83.6 | 70.9 | 82.0 | 73.6 | 87.6 | 78.8 | 79.4 | 7.9% |
| Minutes | | | | | | | | |
| 30 | 92.0 | 89.2 | 90.0 | 87.7 | 98.8 | 93.5 | 91.9 | 4.3% |
| Minutes | | | | | | | | |
| 45 | 94.3 | 93.4 | 92.4 | 89.5 | 101.5 | 96.5 | 94.6 | 4.3% |
| Minutes | | | | | | | | |
| 60 | 96.0 | 95.3 | 93.8 | 93.4 | 103.5 | 98.4 | 96.7 | 3.9% |
| Minutes | | | | | | | | |

Summary of Results (Amlodipine) from the Invention (Water Media)

| | SAMPLE No. | | | | | | | |
|---|---|---|---|---|---|---|---|---|
| TIME | 1 | 2 | 3 | 4 | 5 | 6 | MEAN | RSD |
| 5 Minutes | 38.6 | 23.4 | 35.3 | 25.3 | 23.6 | 24.4 | 28.4 | 23.6% |
| 10 | 65.9 | 45.4 | 61.4 | 48.5 | 44.5 | 51.2 | 52.8 | 16.7% |
| Minutes | | | | | | | | |
| 15 | 82.2 | 63.0 | 78.3 | 67.5 | 61.5 | 69.8 | 70.4 | 11.8% |
| Minutes | | | | | | | | |
| 30 | 99.4 | 86.1 | 94.6 | 84.7 | 79.8 | 90.6 | 89.2 | 8.0% |
| Minutes | | | | | | | | |
| 45 | 105.2 | 94.5 | 100.8 | 92.1 | 83.9 | 95.8 | 95.4 | 7.7% |
| Minutes | | | | | | | | |
| 60 | 107.1 | 99.4 | 104.1 | 95.0 | 88.5 | 96.9 | 98.5 | 6.7% |
| Minutes | | | | | | | | |

Summary of Results from AMLOGARD (Amlodipine Besylate Tablets 5 mg) (Water Media)

| | SAMPLE No. | | | | | | | |
|---|---|---|---|---|---|---|---|---|
| TIME | 1 | 2 | 3 | 4 | 5 | 6 | MEAN | RSD |
| 5 Minutes | 100.8 | 97.9 | 100.4 | 101.6 | 108.9 | 102.0 | 101.9 | 3.6% |
| 10 Minutes | 102.3 | 99.4 | 103.1 | 102.7 | 108.9 | 103.5 | 103.3 | 3.0% |
| 15 Minutes | 102.6 | 100.1 | 105.5 | 105.5 | 111.3 | 103.9 | 104.8 | 3.6% |
| 30 Minutes | 104.2 | 101.6 | 107.8 | 105.0 | 109.7 | 105.0 | 105.6 | 2.7% |
| 45 Minutes | 105.7 | 105.2 | 110.2 | 107.7 | 110.4 | 106.1 | 107.6 | 2.1% |
| 60 Minutes | 106.0 | 104.6 | 110.9 | 106.1 | 110.4 | 106.2 | 107.4 | 2.4% |

Summary of Results from

### Amlogard (Amlodipine

besylate 5 mg tablets)
(0.1 NHCL+0.2% NaCl medial)

| | SAMPLE No. | | | | | | | |
|---|---|---|---|---|---|---|---|---|
| TIME | 1 | 2 | 3 | 4 | 5 | 6 | MEAN | RSD |
| 5 Minutes | 97.1 | 95.9 | 90.3 | 95.9 | 97.5 | 91.5 | 94.7 | 3.2% |
| 10 Minutes | 95.4 | 99.0 | 93.3 | 97.8 | 98.6 | 93.7 | 96.3 | 2.6% |
| 15 Minutes | 96.8 | 98.5 | 95.5 | 100.4 | 98.9 | 95.6 | 97.6 | 2.0% |
| 30 Minutes | 99.9 | 99.5 | 100.2 | 102.3 | 103.1 | 103.4 | 101.4 | 1.7% |
| 45 Minutes | 101.8 | 100.2 | 101.7 | 104.2 | 104.2 | 104.1 | 102.7 | 1.7% |
| 60 Minutes | 102.0 | 100.5 | 102.7 | 104.5 | 105.4 | 104.4 | 103.3 | 1.8% |

### Dissolution of: Atorvastatin+Amlodipine Tablets (80+5) mg

Medium 0.1N HCl (with 0.2% NaCl)
Summary of results (Atrovastatin)

| | SAMPLE No. | | | | | | | |
|---|---|---|---|---|---|---|---|---|
| TIME | 1 | 2 | 3 | 4 | 5 | 6 | MEAN | RSD |
| 5 Minutes | 10.2 | 7.5 | 8.7 | 7.3 | 7.9 | 7.4 | 8.2 | 13.7% |
| 10 | 18.6 | 16.8 | 17.3 | 16.1 | 16.1 | 16.6 | 16.9 | 5.6% |
| Minutes | | | | | | | | |
| 15 | 24.6 | 23.9 | 25.0 | 21.8 | 23.6 | 23.2 | 23.7 | 4.8% |
| Minutes | | | | | | | | |
| 30 | 25.2 | 24.7 | 26.2 | 25.1 | 25.9 | 42.3 | 28.2 | 24.5% |
| Minutes | | | | | | | | |
| 45 | 58.1 | 26.8 | 38.0 | 25.6 | 30.2 | 51.9 | 38.4 | 35.6% |
| Minutes | | | | | | | | |
| 60 | 55.3 | 39.0 | 42.4 | 46.6 | 52.0 | 67.0 | 50.4 | 20.1% |
| Minutes | | | | | | | | |

### Dissolution of Atorvastatin+Amlodipine Tablets (80+5) mg

Medium 0.1N HCl (with 0.2% NaCl)

### Summary of Results (Amlodipine)

| | SAMPLE No. | | | | | | | |
|---|---|---|---|---|---|---|---|---|
| TIME | 1 | 2 | 3 | 4 | 5 | 6 | MEAN | RSD |
| 5 Minutes | 91.4 | 71.1 | 85.1 | 58.4 | 68.1 | 60.9 | 72.5 | 18.2% |
| 10 | 89.8 | 84.6 | 90.6 | 70.1 | 78.9 | 74.6 | 81.4 | 10.2% |
| Minutes | | | | | | | | |
| 15 | 91.8 | 80.7 | 86.6 | 66.8 | 80.6 | 72.0 | 79.8 | 11.5% |
| Minutes | | | | | | | | |
| 30 | 83.5 | 83.9 | 87.3 | 69.7 | 75.5 | 68.3 | 78.0 | 10.3% |
| Minutes | | | | | | | | |
| 45 | 87.9 | 84.1 | 80.9 | 64.8 | 78.2 | 72.1 | 78.0 | 10.8% |
| Minutes | | | | | | | | |
| 60 | 87.3 | 78.7 | 84.5 | 66.8 | 75.5 | 80.3 | 78.9 | 9.2% |
| Minutes | | | | | | | | |

### EXAMPLE 3

A patient on crystalline atorvastatin calcium therapy 20 mg once a day is co-administered 10 mg amlodipine once daily with good therapeutic effect. The patient is subsequently changed to 20 mg once daily amorphous atorvastatin calcium and 10 mg amlodipine without change in the therapeutic effectiveness, while allowing the patient to save a substantial amount in cost of medication.

I/We claim:

1. A method of providing a low cost co-therapy of amlodipine or a pharmaceutically acceptable salt thereof in combination with an amorphous atorvastatin or a pharmaceutically acceptable salt thereof, which co-therapy is sufficiently bioequivalent co-therapy with at least one currently marketed co-therapy of amlodipine or a pharmaceutically acceptable salt thereof and crystalline atorvastatin or a pharmaceutically acceptable salt thereof so as to qualify for an AB rating from The US Food and Drug administration under regulations in force as of the filing date of the present application, comprising providing to a patient amlodipine or a pharmaceutically acceptable salt thereof and a non-crystalline atorvastatin or a pharmaceutically acceptable salt thereof for use in said co-therapy.

2. A method of providing an improved therapeutic regimen to a patient comprising co-therapy of at least one statin and at least one 1,4-dihydropyridine-3,5-dicarboxyldiester.

3. The method of claim 2 wherein the statin is selected from atorvastatin, lovastatin, fluvastatin, pravastatin, rosuvastatin, or simvastatin or a pharmaceutically acceptable salt thereof.

4. The method of claim 2 wherein said stain is amorphous atorvastatin or a pharmaceutically acceptable salt thereof.

5. The method of claim 2 wherein said stain is amorphous atorvastatin hemicalcium.

6. The method of claim 2 wherein the 1,4-dihydropyridine-3,5-dicarboxyldiester is selected from amlodipine, felodipine, isradipine, nicardipine, nifedipine, nimodipine, and nisoldipine or a pharmaceutically acceptable salt thereof.

7. The method of claim 2 wherein the 1,4-dihydropyridine-3,5-dicarboxyldiester is amlodipine.

8. The method of claim 2 in which an additional therapeutic agent is utilized in cotherapy therewith.

9. A fixed combination of at least one statin and at least one 1,4-dihydropyridine-3,5-dicarboxyldiester.

10. The fixed combination of claim 9 wherein the statin is selected from atorvastatin, lovastatin, fluvastatin, pravastatin, rosuvastatin, or simvastatin or a pharmaceutically acceptable salt thereof, with the proviso that the atorvastatin is not in the form of crystalline atorvastatin hemicalcium salt.

11. The fixed combination of claim 9 wherein the statin is atorvastatin or a pharmaceutically acceptable salt thereof, with the proviso that the atorvastatin is not in the form of crystalline atorvastatin hemicalcium salt.

12. The fixed combination of claim 9 wherein the 1,4-dihydropyridine-3,5-dicarboxyldiester is selected from amlodipine, felodipine, isradipine, nicardipine, nifedipine, nimodipine, and nisoldipine or a pharmaceutically acceptable salt thereof.

13. The fixed combination of claim 9 further comprising at least one additional non-1,4-dihydropyridine-3,5-dicarboxyldiester antihypertensive active agent.

14. A co-therapy method of at least one statin active agent, and at least one 1,4-dihydropyridine-3,5-dicarboxydiester active agent alone or optionally in combination with one or more non-1,4-dihydropyridine-3,5-dicarboxydiester antihypertensive active agents and optionally one or more anti-obesity active agents, wherein at least one member selected from said statin and said 1,4-dihydropyridine-3,5-dicarboxydiester is in a fixed combination with at least one other of said active agents in said co-therapy.

15. The co-therapy of claim 14 wherein said fixed combination is selected from
a. said statin active agent plus said 1,4-dihydropyridine-3,5-dicarboxydiester active agent;
b. said statin active agent plus said non-1,4-dihydropyridine-3,5-dicarboxydiester active agent;
c. said statin active agent plus said anti-obesity active agent;
d. said 1,4-dihydropyridine-3,5-dicarboxydiester active agent plus said non-1,4-dihydropyridine-3,5-dicarboxydiester active agent;
e. said 1,4-dihydropyridine-3,5-dicarboxydiester active agent plus said anti-obesity active agent;
f. said statin active agent plus said 1,4-dihydropyridine-3,5-dicarboxydiester active agent plus said non-1,4-dihydropyridine-3,5-dicarboxydiester active agent;
g. said statin active agent plus said 1,4-dihydropyridine-3,5-dicarboxydiester active agent plus said anti-obesity active agent; and
h. said statin active agent plus said 1,4-dihydropyridine-3,5-dicarboxydiester active agent plus said non-1,4-dihydropyridine-3,5-dicarboxydiester active agent plus said anti-obesity active agent;
each of which may be used alone or in free combination with any of (a) said statin active agent, (b) said 1,4-dihydropyridine-3,5-dicarboxydiester active agent (c) said non-1,4-dihydropyridine-3,5-dicarboxydiester active agent, or (d) said anti-obesity active agent not contained in said fixed combination, or two fixed combinations (a)-(e) above may be used in free combination with each other.

16. A formulation of at least a statin comprising
a. an effective amount of a statin or a pharmaceutically acceptable salt thereof;
b. vitamin E TPGS;
c. croscarmellose sodium;
d. microcrystalline cellulose;
e. lactose monohydrate;
f. sodium starch glycollate;
g. magnesium stearate;
h. optionally a film coat; and
i. optionally an effective amount of one or more additional active agents selected from the group consisting of
A. 1,4-dihydropyridine-3,5-dicarboxydiester antihypertensive active agents;
B. non-1,4-dihydropyridine-3,5-dicarboxydiester antihypertensive active agents;
C. anti-obesity active agents.

17. The formulation of claim 16 wherein
a. said croscarmellose, said vitamin E TPGS are intragranular with said statin;
(b) optionally a portion of said microcrystalline cellulose is intragranular with said statin; and
(c) optionally one or more of said optional additional active agents are intragranular with said statin.

18. The formulation of claim 17 wherein at least one of said 1,4-dihydropyridine-3,5,-dicarboxydiester antihypertensive active agents is present.

19. The formulation of claim 18 wherein said 1,4-dihydropyridine-3,5,-dicarboxydiester antihypertensive active agents is present intragranularly with said statin.

20. The formulation of claim 16 wherein comprising at least atorvastatin as said statin active agent and amlodipine as said 1,4-dihydropyridine-3,5,-dicarboxydiester antihypertensive active agent.
